(19) United States
(12) Patent Application Publication
(10) Pub. No.: US 2024/0329242 A1
(43) Pub. Date: Oct. 3, 2024

(54) ULTRASOUND PROBE AND ULTRASOUND DIAGNOSIS APPARATUS

(71) Applicant: Konica Minolta, Inc., Tokyo (JP)

(72) Inventors: Kenji SUZUKI, Tokyo (JP); Yuta NAKAYAMA, Ichikawa-shi (JP)

(21) Appl. No.: 18/610,324

(22) Filed: Mar. 20, 2024

(30) Foreign Application Priority Data
Mar. 29, 2023 (JP) 2023-052575

(51) Int. Cl. G01S 15/89; G01S 7/52

(52) U.S. Cl. CPC G01S 15/8925; G01S 7/52079; G01S 15/8952

(57) ABSTRACT

An ultrasound probe including a PMUT array including a plurality of channels and including a plurality of three or more PMUT cells within each of the channels, in which piezoelectric elements of the plurality of PMUT cells are electrically connected to each other in a series/parallel mixed connection in which parallel connections and series connections are mixed.

### CROSS-REFERENCE TO RELATED APPLICATIONS

The present invention claims priority under 35 U.S.C. § 119 to Japanese Application, 2023-052575, filed on Mar. 29, 2023, the entire contents of which being incorporated herein by reference.

### BACKGROUND OF THE INVENTION

### Technical Field

The present invention relates to an ultrasound probe and an ultrasound diagnosis apparatus.

### Description of Related Art

Ultrasound diagnosis in which a state of the heart or the fetus can be obtained as an ultrasonic image with simple operation of placing an ultrasound probe to a body surface or from a body cavity of a patient, has high safety, and thus, examinations can be repeatedly performed. Ultrasonic image data is obtained by ultrasounds being transmitted from the ultrasound probe having a piezoelectric element to a subject, the reflected ultrasounds being received by the ultrasound probe, and various kinds of processing being performed on the received signals.

Further, an ultrasound probe using a piezoelectric micromachined ultrasonic transducer (PMUT) is known. The PMUT is a transducer (ultrasound probe) based on bending motion of a thin film binding with a piezoelectric thin film by a micro electro mechanical systems (MEMS). For example, an ultrasonic transducer (see JP 6933082 B) is known in which a plurality of adjacent pMUT cells included in one transmission/reception channel (CH) are connected in parallel or in series.

Further, a micromachined ultrasonic transducer array (see JP 2016-503312 A) having a plurality of electrode rails having a plurality of transducer elements with different resonance frequencies within an MUT array is known. The electrode rails are provided for each channel, and the transducer elements in each channel are electrically connected in parallel.

For example, in a case of an ultrasound probe of a one-dimensional (1D) array, a frequency band and an element area (a minor axis width, a major axis length) are generally determined from a target to be imaged (a depth, a size). In a case of a 1D array using the PMUT, approximately several hundreds of PMUT cells are included in the element area and are electrically connected in parallel.

Typically, the PMUT is constituted with a PMUT array in which a plurality of PMUT cells by a piezoelectric thin film, a vibrating membrane and a vibrating membrane support structure (back cavity) are arranged. The piezoelectric thin film is manufactured on a silicon wafer using a sputtering method, or the like, and has a constant thickness. The vibrating membrane has a constant film thickness that is a film thickness of a device layer of a substrate to be used, for example, a silicon on insulator (SOI) substrate. A resonance frequency of the PMUT cells can be controlled by a diameter of the vibrating membrane, and a desired frequency band can be obtained by changing a distribution of the diameter.

Electric characteristics (impedance characteristics) of the piezoelectric element are generally represented well with an electrostatic capacitance component and a resistance component (except a resonance point). The electrostatic capacitance is generally determined from relative permittivity, a film thickness and a diameter of the piezoelectric thin film of the PMUT cells. Further, if a design frequency band is determined once, the diameter of the PMUT is generally determined so that the resonance frequency thereof becomes in the vicinity of the design frequency band, and it is very difficult to control the electrostatic capacitance.

For example, a wireless ultrasound probe in which a complementary metal-oxide semiconductor (CMOS) circuit is closely connected to the PMUT cells will be assumed. In a case of the PMUT cells only connected in parallel, the electrostatic capacitance becomes too large. While it is possible to cancel the electrostatic capacitance by adding an inductor circuit, addition of the circuit increases a size and weight of the ultrasound probe, which significantly degrades usability. Further, addition of the circuit increases cost.

Inversely, in a case of the PMUT cells only connected in series, the electrostatic capacitance becomes too small. Further, as a result of a voltage to be applied to the PMUT cells upon transmission being decreased, transmission sensitivity also decreases.

It is therefore difficult to adjust the electrostatic capacitance to be appropriate electrostatic capacitance in both parallel and series connection forms as in the ultrasonic transducer or the micromachined ultrasonic transducer array in the related art described above.

### SUMMARY OF THE INVENTION

An object of the present invention is to appropriately control electrostatic capacitance of a piezoelectric element in accordance with a connection form between an ultrasound probe and parts in the subsequent stage (a transmitter/receiver, a cable) and achieve impedance matching.

To achieve at least one of the abovementioned objects, according to an aspect of the present invention, an ultrasound probe reflecting one aspect of the present invention includes:
a PMUT array including a plurality of channels and including a plurality of three or more PMUT cells within each of the channels,
in which piezoelectric elements of the plurality of PMUT cells are electrically connected to each other in a series/parallel mixed connection in which parallel connections and series connections are mixed.

### BRIEF DESCRIPTION OF THE DRAWINGS

The advantages and features provided by one or more embodiments of the invention will become more fully understood from the detailed description given hereinbelow and the appended drawings which are given by way of illustration only, and thus are not intended as a definition of the limits of the present invention, wherein:

FIG. 1 is a block diagram illustrating a functional configuration of a first ultrasound diagnosis apparatus of an embodiment of the present invention,

FIG. 2 is a block diagram illustrating a functional configuration of a second ultrasound diagnosis apparatus according to the embodiment,

FIG. 3A is a plan view of a PMUT array,

FIG. 3B is a plan view of a partial region of the PMUT array in FIG. 3A,

FIG. 4 is a cross-sectional view of the PMUT array in a parallel connection,

FIG. 5 is a cross-sectional view of the PMUT array in a series connection,

FIG. 6A is a schematic cross-sectional view of the PMUT array,

FIG. 6B is a schematic cross-sectional view of the PMUT array,

FIG. 6C is a schematic cross-sectional view of the PMUT array,

FIG. 7A is a plan view of the PMUT array,

FIG. 7B is a plan view of the PMUT array,

FIG. 7C is a plan view of the PMUT array,

FIG. 8A is a circuit diagram of the PMUT array in an all parallel connection,

FIG. 8B is a circuit diagram of the PMUT array in an all series connection,

FIG. 9A is a circuit diagram of the PMUT array in a first series/parallel mixed connection,

FIG. 9B is a circuit diagram of the PMUT array in a second series/parallel mixed connection,

FIG. 10 is a circuit diagram of the PMUT array in a third series/parallel mixed connection,

FIG. 11 is a schematic circuit diagram of the PMUT array in a series/parallel mixed connection,

FIG. 12 is a schematic plan view illustrating PMUT cells in a PMUT array in an application example,

FIG. 13 is a schematic plan view illustrating blocks of the PMUT array in the application example; and

FIG. 14 is a schematic view illustrating a PMUT array in a modified example.

### DETAILED DESCRIPTION

Hereinafter, one or more embodiments of the present invention will be described with reference to the drawings. However, the scope of the invention is not limited to the disclosed embodiments.

### Embodiment

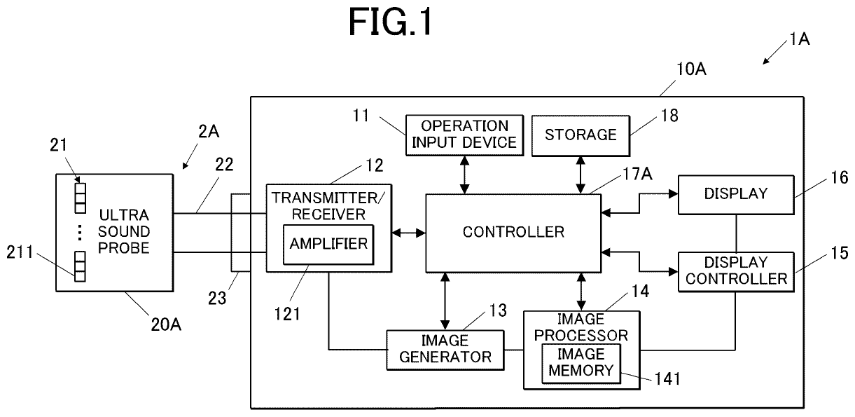

An embodiment according to the present invention will be described with reference to FIG. 1 to FIG. 11. It is assumed in the present embodiment that two ultrasound diagnosis apparatuses 1A and 1B are used. The ultrasound diagnosis apparatuses 1A and 1B, which are installed in a medical facility such as a hospital and used by a user such as a doctor and a technologist, generate ultrasonic image data of a subject such as a living body of a patient.

First, an apparatus configuration of the ultrasound diagnosis apparatus 1A of the present embodiment will be described with reference to FIG. 1. FIG. 1 is a block diagram illustrating a functional configuration of the ultrasound diagnosis apparatus 1A of the present embodiment.

As illustrated in FIG. 1, the ultrasound diagnosis apparatus 1A includes an ultrasound diagnosis apparatus body 10A and an ultrasound probe 2A. The ultrasound diagnosis apparatus 1A is an ultrasound diagnosis apparatus in which a transmitter/receiver 12 which will be described later is located on the ultrasound diagnosis apparatus body 10A side.

The ultrasound probe 2A transmits ultrasounds (transmission ultrasounds) to the inside of the subject and receives reflected waves (reflected ultrasounds: echo) reflected inside the subject. The ultrasound diagnosis apparatus body 10A is connected to the ultrasound probe 2A. The ultrasound diagnosis apparatus body 10A causes the ultrasound probe 2A to transmit transmission ultrasounds to the subject by transmitting a drive signal that is an electrical signal to the ultrasound probe 2A. Then, the ultrasound diagnosis apparatus body 10A receives a reception signal that is an electrical signal generated at the ultrasound probe 2A in accordance with the reflected ultrasounds received at the ultrasound probe 2A from the inside of the subject. Then, the ultrasound diagnosis apparatus body 10A generates an image of an internal state of the inside of the subject as ultrasonic image data on the basis of the reception signal.

The ultrasound probe 2A includes a head 20A, a cable 22 and a connector 23. The head 20A, which is a distal end portion that is located on a distal end side of the ultrasound probe 2A and transmits/receives ultrasounds, includes a PMUT array 21 including a plurality of PMUT cells 211 on the distal end side. The ultrasound probe 2A places the PMUT array 21 to the subject to transmit and receive ultrasounds. The PMUT cells 211 are piezoelectric elements (transducer elements) that are constituted with MEMS and transmit/receive ultrasounds. It is assumed that a plurality of the PMUT cells 211 are, for example, arranged in a one-dimensional array in an azimuth direction (an azimuth direction, a scanning direction) and are also arranged in an elevation direction perpendicular to the azimuth direction. However, a plurality of the PMUT cells 211 may be arranged in a one-dimensional array only in the azimuth direction. Further, the number of the PMUT cells 211 in the PMUT array 21 can be arbitrarily set. Still further, it is assumed in the present embodiment that scanning with ultrasounds is performed in a linear scanning scheme using an electronic scanning probe using a linear scanning scheme as the ultrasound probe 2A. However, probes using other scanning schemes such as a probe using a convex scanning scheme and a probe using a sector scanning scheme can be also employed as the ultrasound probe 2A. The PMUT array 21 will be described in detail later.

The cable 22 has one end electrically connected to the head 20A and the other end electrically connected to the connector 23. It is assumed here that the cable 22 is a coaxial cable having impedance close to 50[Ω]. More specifically, it is assumed that the cable 22 is, for example, a coaxial cable having impedance Z=50 to 75[Ω] and electrostatic capacitance of 60 to 100 [pF/m]. The ultrasound probe 2A using the cable 22 is used in the ultrasound diagnosis apparatus (system) in the related art although the ultrasound probe 2A using the cable 22 is relatively expensive.

The connector 23 is a plug-type connector electrically connected to the cable 22 and connected to the ultrasound diagnosis apparatus body 10A. The connector 23 is detachably electrically connected to a receptacle-type connector (not illustrated) of the ultrasound diagnosis apparatus body 10A.

The ultrasound diagnosis apparatus body 10A includes, for example, an operation input device 11, the transmitter/receiver 12, an image generator 13, an image processor 14, a display controller 15, a display 16, a controller 17A and a storage 18.

The operation input device 11 accepts an operation input from the user. The operation input device 11 is, for example, an operation input device for inputting a command that gives an instruction to start a diagnosis, various kinds of image parameters for displaying ultrasonic image data, and the like, on the display 16, and the like. The operation input device 11 includes various kinds of switches, buttons, a trackball, a mouse, a keyboard, a touch pad, and the like, and outputs an operation signal to the controller 17A. Note that the operation input device 11 may include a touch panel that is provided on a display panel of the display 16 and accepts a touch input from the operator.

The transmitter/receiver 12 is a circuit that supplies a drive signal that is an electrical signal to the ultrasound probe 2A to generate transmission ultrasounds in accordance with control by the controller 17A as a transmitter. Further, the transmitter/receiver 12 includes, for example, a clock generation circuit, a delay circuit and a pulse generation circuit. The clock generation circuit is a circuit that generates a clock signal that determines a transmission timing and a transmission frequency of the drive signal. The delay circuit is a circuit that sets a delay period for each of individual paths corresponding to the respective PMUT cells and delays transmission of the drive signal by the set delay period. The delay circuit focuses transmission beams constituted with transmission ultrasounds. The pulse generation circuit is a circuit for generating a pulse signal as the drive signal at predetermined intervals. The transmitter/receiver 12 constituted as described above, for example, drives successive part among the plurality of PMUT cells 211 arranged in the ultrasound probe 2A to generate transmission ultrasounds. Then, the transmitter/receiver 12 performs scanning by shifting PMUT cells to be driven in the azimuth direction every time the transmission ultrasounds are generated.

Further, the transmitter/receiver 12 functions as a circuit that receives a reception signal that is an electrical signal from the ultrasound probe 2A in accordance with control by the controller 17A as a receiver. The transmitter/receiver 12 includes, for example, an amplifier, an A/D conversion circuit and a phasing addition circuit. The amplifier is a circuit for amplifying the reception signal with an amplification factor set in advance for each of the individual paths corresponding to the respective PMUT cells 211. The A/D conversion circuit is a circuit for converting the amplified reception signal from an analog signal to a digital signal. The phasing addition circuit is a circuit that provides a delay period for each of the individual paths corresponding to the respective PMUT cells to the A/D converted reception signal to adjust a time phase and performs addition (phasing addition). The phasing addition circuit generates sound ray data through the phasing addition.

Further, the transmitter/receiver 12 includes an amplifier 121 that amplifies a signal. The amplifier 121 is, for example, a bipolar amplifier having impedance Z of approximately 200[Ω]. The bipolar amplifier is relatively expensive and has characteristics of high power consumption, a large amount of heat generation, and a large area. The ultrasound diagnosis apparatus body 10A is connected to a commercial power supply (not illustrated) and is driven with power supplied from the commercial power supply, and thus, a bipolar amplifier with high power consumption is used as the amplifier 121.

The image generator 13 can generate brightness (B) mode image data including pixels having brightness values as reception energy by performing envelope detection processing, logarithmic compression, and the like, on the sound ray data from the transmitter/receiver 12 and converting brightness by adjusting a dynamic range and a gain in accordance with control by the controller 17A. In other words, the B mode image data represents strength of the reception signal with brightness. The image generator 13 may be a generator capable of generating ultrasonic image data in other scanning modes such as an amplitude (A) mode, a motion (M) mode and a scanning mode using a Doppler method (such as a color Doppler mode and a pulsed wave doppler (PWD)) other than the B mode image data for which the scanning mode (image mode) is the B mode.

The image processor 14 performs image processing on the B mode image data output from the image generator 13 in accordance with various kinds of image parameters that are being set in accordance with control by the controller 17A. Further, the image processor 14 includes an image memory 141 constituted with a semiconductor memory such as a dynamic random access memory (DRAM). The image processor 14 stores the B mode image data subjected to the image processing in the image memory 141 in frame units in accordance with control by the controller 17A. The image data in frame units may be referred to as ultrasonic image data or frame image data. The image processor 14 outputs the image data generated as described above to the display controller 15 in accordance with control by the controller 17A.

The display controller 15 performs coordinate conversion, or the like, on the ultrasonic image data received from the image processor 14 to convert the ultrasonic image data into an image signal for display and outputs the image signal for display to the display 16 in accordance with control by the controller 17A.

As the display 16, a display device such as a liquid crystal display (LCD), a cathode-ray tube (CRT) display, an organic electronic luminescence (EL) display, an inorganic EL display and a plasma display can be applied. The display 16 displays a still image or a moving image of the ultrasonic image data on a display screen in accordance with the image signal output from the display controller 15 in accordance with control by the controller 17A.

The controller 17A includes, for example, a central processing unit (CPU), a random access memory (RAM) and a storage. The controller 17A reads out and loads to the RAM various kinds of programs stored in the storage and controls each component of the ultrasound diagnosis apparatus 1A in accordance with the loaded programs. The storage, which is constituted with a non-volatile memory such as a semiconductor, stores a system program supporting the ultrasound diagnosis apparatus 1A and various kinds of processing programs executable on the system program, and various kinds of data such as various kinds of tables. These programs are stored in a form of a computer readable program code, and the CPU sequentially executes operation in accordance with the program code. The RAM forms a work area for temporarily storing various kinds of programs to be executed by the CPU and data related to the programs.

In particular, the controller 17A accepts operation information regarding display of the ultrasonic image from the user via the operation input device 11. Then, the controller 17A causes the transmitter/receiver 12 to generate a drive signal and output the drive signal to the ultrasound probe 2A on the basis of the operation information. Then, the controller 17A causes the transmitter/receiver 12 to receive the reception signal input from the ultrasound probe 2A and generate sound ray data. Then, the controller 17A causes the image generator 13 to generate B mode image data from the sound ray data. Then, the controller 17A causes the B mode image data to be displayed at the display 16 as a B mode image via the image processor 14 and the display controller 15.

The storage 18, which is constituted with a hard disk drive (HDD), a solid state drive (SSD), or the like, stores data such as the ultrasonic image data.

In the ultrasound diagnosis apparatus 1A, the PMUT cells 211 are required to achieve impedance matching (Z=approximately 50 to 75[Ω]) and electrostatic capacitance matching (approximately 100 to 200 [pF]) with the cable 22.

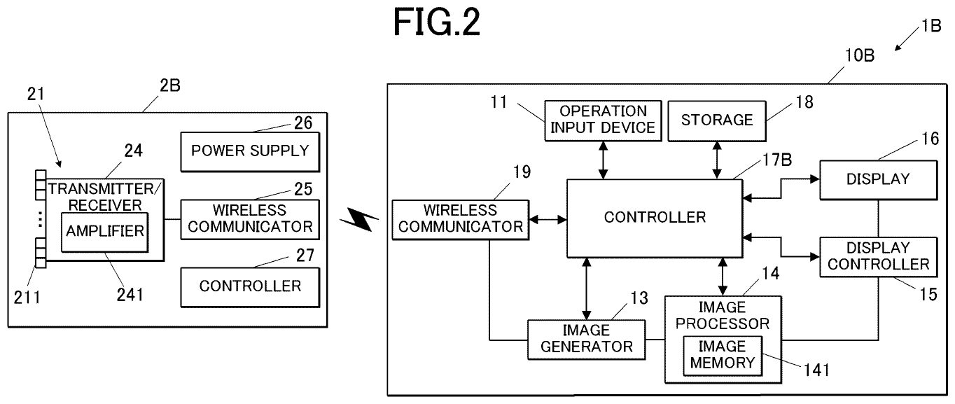

An apparatus configuration of the ultrasound diagnosis apparatus 1B of the present embodiment will be described next with reference to FIG. 2. FIG. 2 is a block diagram illustrating a functional configuration of the ultrasound diagnosis apparatus 1B of the present embodiment.

As illustrated in FIG. 2, the ultrasound diagnosis apparatus 1B includes an ultrasound diagnosis apparatus body 10B and an ultrasound probe 2B. The ultrasound diagnosis apparatus 1B is an ultrasound diagnosis apparatus in which a transmitter/receiver 24 which will be described later is located on the ultrasound probe 2B side. In the ultrasound diagnosis apparatus 1B, portions similar to those of the ultrasound diagnosis apparatus 1A are denoted by the same reference numerals, and description thereof will be omitted.

The ultrasound diagnosis apparatus body 10B includes, for example, the operation input device 11, the image generator 13, the image processor 14, the display controller 15, the display 16, a controller 17B, the storage 18 and a wireless communicator 19. The ultrasound probe 2B includes the PMUT array 21, a transmitter/receiver 24, a wireless communicator 25, a power supply 26 and a controller 27.

The controller 17B has a similar configuration as that of the controller 17A and controls each component of the ultrasound diagnosis apparatus 1B. In particular, the controller 17B accepts operation information regarding display of the ultrasonic image from the user via the operation input device 11. Then, the controller 17B generates information for generating a drive signal on the basis of the operation information and transmits the information to the controller 27 via the wireless communicators 19 and 25. Then, the controller 17B causes the transmitter/receiver 24 to generate a drive signal and output the drive signal to the PMUT array 21 by the controller 27. Then, the controller 17B causes the transmitter/receiver 24 to receive a reception signal input from the PMUT array 21 and generate sound ray data by the controller 27. Then, the controller 17B causes the generated sound ray data to be transmitted to the wireless communicator 19 via the wireless communicator 25 by the controller 27. Then, the controller 17B causes the image generator 13 to generate B mode image data from the sound ray data received at the wireless communicator 19. Then, the controller 17B causes the B mode image data to be displayed at the display 16 as a B mode image via the image processor 14 and the display controller 15.

The wireless communicator 19 is a wireless communicator that performs wireless communication with (the wireless communicator 25 of) the ultrasound probe 2b using a predetermined wireless communication scheme. The predetermined wireless communication scheme is, for example, an ultra wide band (UWB), but is not limited to this wireless communication scheme. The controller 17B transmits/receives information to/from the ultrasound probe 2B via the wireless communicator 19. Further, the wireless communicator 19 outputs the sound ray data received from the ultrasound probe 2B to the image generator 13 in accordance with control by the controller 17B.

The transmitter/receiver 24 is a circuit for supplying a drive signal that is an electrical signal to the PMUT array 21 to generate a transmission ultrasounds in accordance with control by the controller 27 as a transmitter in a similar manner to the transmitter/receiver 12. Further, the transmitter/receiver 24 functions as a circuit that receives a reception signal that is an electrical signal from the PMUT array 21 in accordance with control by the controller 27 as a receiver in a similar manner to the transmitter/receiver 12. The transmitter/receiver 24 generates sound ray data from the reception signal.

Further, the transmitter/receiver 24 is positioned so as to be connected (closely connected) in the vicinity of the PMUT array 21 within a chassis of the ultrasound probe 2B. It is therefore not necessary to provide an expensive coaxial cable between the PMUT array 21 and the transmitter/receiver 24.

Further, the transmitter/receiver 24 includes an amplifier 241 that amplifies a signal. It is assumed that the amplifier 241 is a CMOS amplifier having impedance Z=2 [kΩ] and electrostatic capacitance of approximately 0.1 to 5 [pF]. Note that the amplifier 241 is not limited to this and may be, for example, a CMOS amplifier having electrostatic capacitance of 0.02 to 10 [pF]. The CMOS amplifier is relatively inexpensive and has characteristics of low power consumption, a small amount of heat generation, and a small area. The ultrasound probe 2B is driven with power supplied from the power supply 26 not connected to a commercial power supply, and thus, the CMOS amplifier with low power consumption is used as the amplifier 241. Further, by using the CMOS amplifier, a surface temperature of the ultrasound probe 2B that is in contact with the subject can be lowered.

The wireless communicator 25 is a wireless communicator that performs wireless communication with (the wireless communicator 19 of) the ultrasound diagnosis apparatus body 10B using a predetermined wireless communication scheme such as UWB. The controller 27 transmits/receives information to/from the ultrasound diagnosis apparatus body 10B via the wireless communicator 25. Further, the wireless communicator 25 outputs information for generating a drive signal received from the ultrasound diagnosis apparatus body 10B to the transmitter/receiver 24 in accordance with control by the controller 27. Still further, the wireless communicator 25 transmits the sound ray data generated at the transmitter/receiver 24 to the ultrasound diagnosis apparatus body 10B in accordance with control by the controller 27.

The power supply 26 is constituted with a battery of a primary cell or a secondary cell and supplies power to each component of the ultrasound probe 2B.

The controller 27 controls each component of the ultrasound probe 2B mainly in accordance with control by the controller 17B. The controller 27 is constituted with a CPU, a RAM, a storage, a control circuit, and the like.

In the ultrasound diagnosis apparatus 1B, it is necessary to change impedance and electrostatic capacitance of the PMUT cells 211 by the amplifier 241 (CMOS amplifier).

Further, the ultrasound diagnosis apparatus 1B has a configuration in which the ultrasound diagnosis apparatus body 10B performs communication with the ultrasound probe 2B through wireless communication by the wireless communicators 19 and 25, but this is not restrictive. The ultrasound diagnosis apparatus 1B may have a configuration in which the ultrasound diagnosis apparatus body 10B performs communication with the ultrasound probe 2B through wired communication using a universal serial bus (USB) cable, or the like.

Note that concerning components included in the ultrasound diagnosis apparatuses 1A and 1B, functions of part or all of the respective functional blocks can be implemented as a hardware circuit such as an integrated circuit. The integrated circuit is, for example, a large scale integration (LSI), and the LSI is sometimes referred to as an integrated circuit (IC), a system LSI, a super LSI or an ultra LSI in accordance with a difference in an integration degree. Further, an integration method is not limited to the LSI and may be implemented with a dedicated circuit or a general-purpose processor, or a reconfigurable processor that can reconfigure connections and setting of circuit cells inside a field programmable gate array (FPGA) and the LSI may be utilized. Still further, functions of part or all of the respective functional blocks may be executed by software. In this case, this software is stored in one or more storage media such as a ROM, optical disks, hard disks, or the like, and this software is executed by an arithmetic processor.

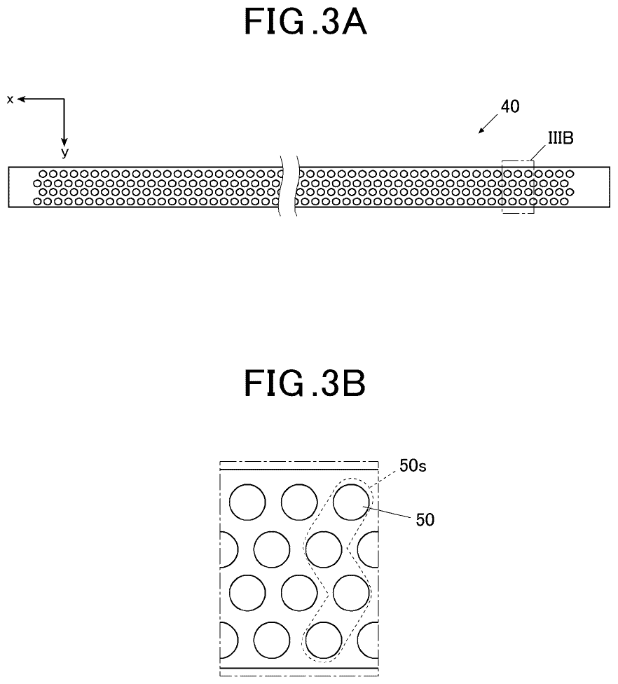

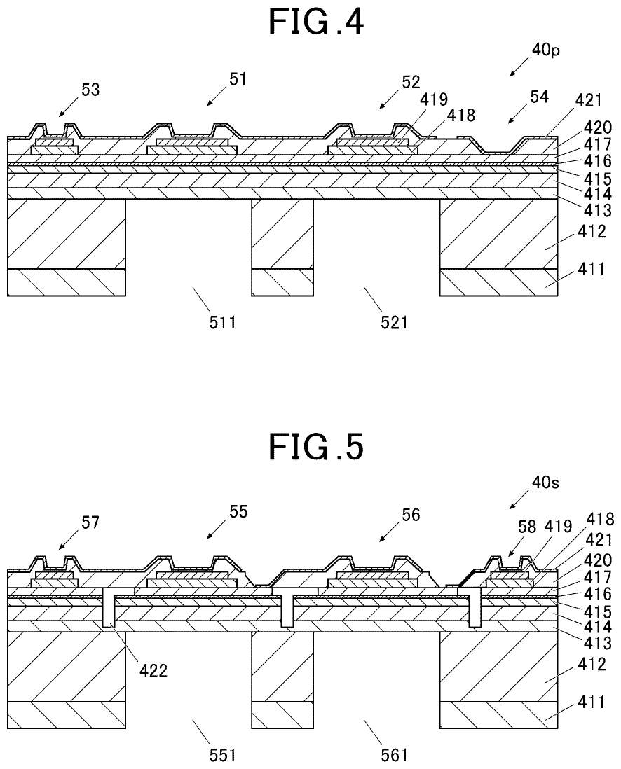

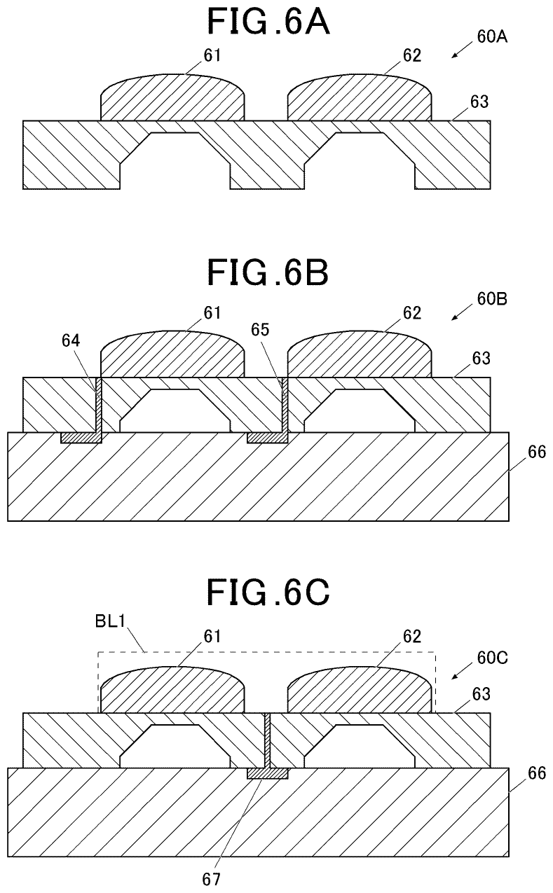

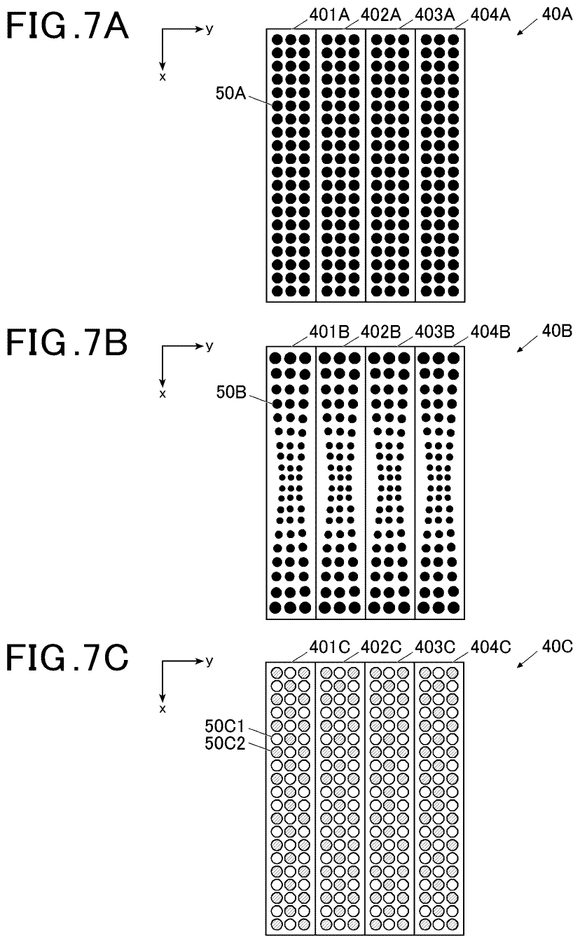

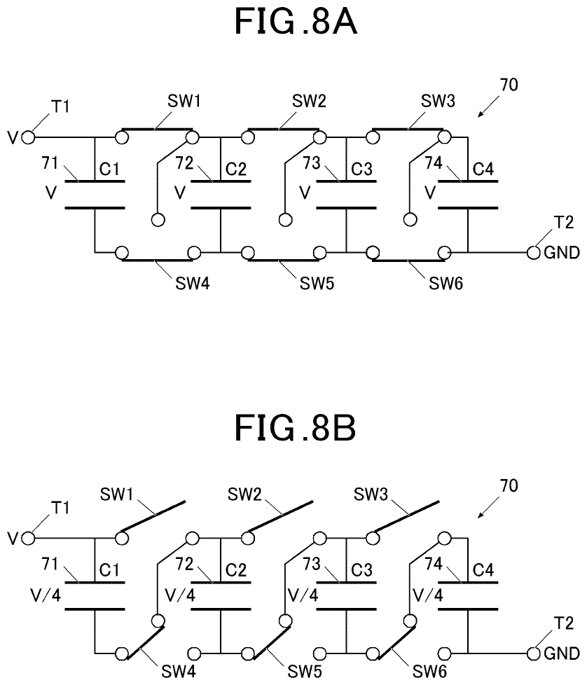

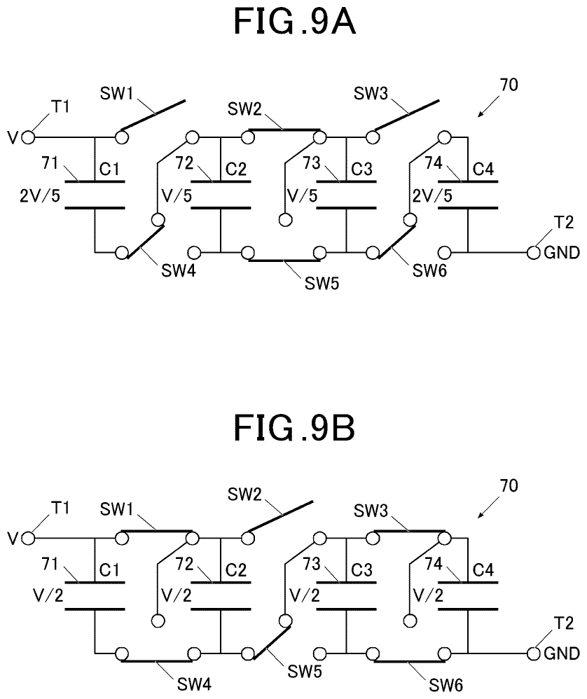

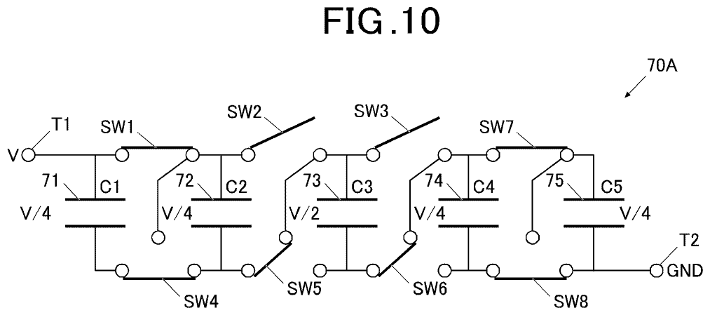

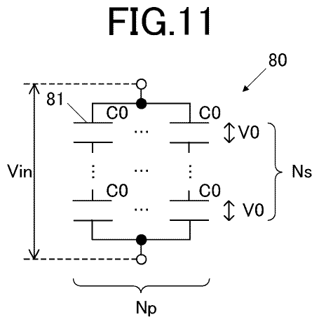

A configuration of the PMUT array 21 will be described next with reference to FIG. 3A to FIG. 11. FIG. 3A is a plan view of a PMUT array 40. FIG. 3B is a plan view of a partial region IIIB of the PMUT array 40. FIG. 4 is a cross-sectional view of a PMUT array 40p in a parallel connection. FIG. 5 is a cross-sectional view of a PMUT array 40s in a series connection. FIG. 6A is a schematic cross-sectional view of a PMUT array 60A. FIG. 6B is a schematic cross-sectional view of a PMUT array 60B. FIG. 6C is a schematic cross-sectional view of a PMUT array 60C. FIG. 7A is a plan view of a PMUT array 40A. FIG. 7B is a plan view of a PMUT array 40B. FIG. 7C is a plan view of a PMUT array 40C. FIG. 8A is a circuit diagram of a PMUT array 70 in an all parallel connection. FIG. 8B is a circuit diagram of the PMUT array 70 in an all series connection. FIG. 9A is a circuit diagram of the PMUT array 70 in a first series/parallel mixed connection. FIG. 9B is a circuit diagram of the PMUT array 70 in a second series/parallel mixed connection. FIG. 10 is a circuit diagram of a PMUT array 70A in a third series/parallel mixed connection. FIG. 11 is a schematic circuit diagram of a PMUT array in a series/parallel mixed connection.

As illustrated in FIG. 3A, the PMUT arrays 21 of the ultrasound probes 2A and 2B have, for example, a configuration of the PMUT array 40. In the PMUT array 40, a plurality of PMUT cells 50 corresponding to the PMUT cells 211 are arranged on an xy plane. Here, an x axis is an elevation direction of the PMUT array 40. A y axis is an azimuth direction (azimuth direction) of the PMUT array 40. Meaning of the directions of the x axis and the y axis is similar in other drawings.

The PMUT array 40 includes a plurality of channels that is a unit of controlling transmission/reception of ultrasounds. A plurality of PMUT cells 50 within one channel are connected in a series/parallel mixed connection in which parallel connections and series connections are mixed. FIG. 3B illustrates an enlarged plan view of the partial region IIIB of the PMUT cells 50 in FIG. 3A. In the partial region IIIB, a PMUT cell group 50s within one channel includes four PMUT cells 50 connected in series in the x axis direction (elevation direction). Further, it is assumed that a plurality of PMUT cell groups 50s are connected in parallel within one channel. In this manner, at least three PMUT cells 50 are included within one channel to enable a series/parallel mixed connection.

Subsequently, the PMUT array 40p in which PMUT cells 51 and 52 are connected in parallel will be described as an example of the PMUT array 21 with reference to FIG. 4. Here, while the PMUT array 40p including two PMUT cells will be described to simplify description, the number of PMUT cells is not limited to this.

In the PMUT array 40p, an SiO₂ layer 411, an Si layer 412, an SiO₂ layer 413, an Si layer 414, an SiO₂ layer 415, a Ti layer 416, a lower electrode 417, a piezoelectric thin film 418, an upper electrode 419, an insulating film 420 and an upper extraction electrode 421 are sequentially laminated from a lower side to an upper side.

The SiO₂ layers 411, 413 and 415 are layers of SiO₂ that is an insulator. The Si layers 412 and 414 are layers of Si that is a semiconductor. While the Ti layer 416 is a layer of Ti that is a metal and is part of a lower electrode of the piezoelectric thin film 418, the Ti layer 416 is provided to prevent the SiO₂ layer 415 from coming into direct contact with the lower electrode 417. The lower electrode 417 is a lower electrode of the piezoelectric thin film 418 and is a layer of Pt that is a metal. The piezoelectric thin film 418 is a body of the piezoelectric element and is a layer of lead zirconate titanate (PZT) that is a piezoelectric body. The upper electrode 419 is an upper electrode of the piezoelectric thin film 418 and is a layer of Pt, or the like, that is a metal. The insulating film 420 is the lower electrode 417 and a protective insulating film of the piezoelectric thin film 418 and the lower electrode 417 and is a layer of parylene, or the like, that is an insulator. The upper extraction electrode 421 is an electrode electrically connected to the upper electrode 419 and to be extracted and is a layer of Au, or the like, that is a metal.

In the PMUT array 40p, the PMUT cells 51 and 52, an upper electrode pad 53 and a lower electrode pad 54 are formed. The PMUT cells 51 and 52 are piezoelectric elements that transmit/receive ultrasounds, and the lower electrode 417 and the upper electrode 419 are electrically connected so that the piezoelectric thin films 418 are parallel to each other. The PMUT cell 51 includes a cavity 511 formed by removing films of the SiO₂ layer 411 and the Si layer 412. The Ti layer 416 to the upper extraction electrode 421 of the PMUT cell 51 are formed on the SiO₂ layer 413 to the Si layer 412 in a film shape above the cavity 511. In a similar manner, the PMUT cell 52 includes a cavity 521. Above the cavity 521, the Ti layer 416 to the upper extraction electrode 421 of the PMUT cell 52 are formed.

The upper electrode pad 53 is an electrode pad electrically connected to the upper electrode 419. The lower electrode pad 54 is an electrode pad electrically connected to the lower electrode 417.

Note that in FIG. 4, illustration of portions (such as a protective layer and an acoustic lens) on a surface side of the PMUT array 21 (such as the piezoelectric thin film 418) of the ultrasound probes 2A and 2B and portions (such as a bucking member) on a back surface side of the PMUT array 21 is omitted. Also, in FIG. 5 which will be described later, illustration of the portions on the surface side and portions on the back surface side of the PMUT array 21 will be omitted.

Subsequently, the PMUT array 40s in which the PMUT cells 55 and 56 are connected in series will be described as an example of the PMUT array 21 with reference to FIG. 5. In the PMUT array 40s, the SiO₂ layer 411, the Si layer 412, the SiO₂ layer 413, the Si layer 414, the SiO₂ layer 415, the Ti layer 416, the lower electrode 417, the piezoelectric thin film 418, the upper electrode 419, the insulating film 420 and the upper extraction electrode 421 are sequentially laminated from a lower side to an upper side.

In the PMUT array 40s, the PMUT cells 55 and 56, the upper electrode pad 57 and the lower electrode pad 58 are formed. The PMUT cells 55 and 56 are piezoelectric elements that transmit/receive ultrasounds, and the lower electrode 417 and the upper electrode 419 are electrically connected so that the piezoelectric thin films 418 become parallel to each other. The PMUT cell 55 includes a cavity 551 formed by removing films of the SiO₂ layer 411 and the Si layer 412. The Ti layer 416 to the upper extraction electrode 421 of the PMUT cell 55 are formed on the SiO₂ layer 413 to the Si layer 412 in a film shape above the cavity 551. In a similar manner, the PMUT cell 56 includes a cavity 561. The Ti layer 416 to the upper extraction electrode 421 of the PMUT cell 56 are formed above the cavity 561.

Further, in the PMUT array 40s, insulating trenches 422 are formed. The insulating trenches 422 are respectively disposed between the PMUT cells 55 and 56, and the upper electrode pad 57 and the lower electrode pad 58 and float (separate) the respective lower electrodes 417.

Subsequently, a configuration where a connection of the PMUT cells cannot be switched and a configuration where a connection of the PMUT cells can be switched will be described as an example of the PMUT array 21 with reference to FIG. 6A. The PMUT array including two PMUT cells will be described here to simplify description, but this is not restrictive.

As illustrated in FIG. 6A, the PMUT array 60A will be described as an example of the PMUT array 21. The PMUT array 60A includes PMUT cells 61 and 62 and a MEMS 63. The PMUT cells 61 and 62 are bodies of the PMUT cells 211. The PMUT cells 61 and 62 are portions corresponding to the Ti layer 416 to the upper extraction electrode 421 of the PMUT cells 51, 52, 55 and 56 of the PMUT arrays 40p and 40s in FIG. 4 and FIG. 5. The MEMS 63 is part of the PMUT cell 211. The MEMS 63 is a portion corresponding to the SiO₂ layer 411 to the upper extraction electrode 421 of the PMUT cells 51, 52, 55 and 56 of the PMUT arrays 40p and 40s in FIG. 4 and FIG. 5.

The PMUT array 60A has a configuration in which all structures of the PMUT cells 61 and 62 including series/parallel structures of the PMUT cells 61 and 62 and the MEMS 63 are fabricated by a MEMS substrate. The PMUT array 60A does not include a circuit switching element, and thus, a connection of the PMUT cells 61 and 62 cannot be switched between a series connection and a parallel connection. However, the PMUT array 60A has only a structure of the MEMS substrate, which is a simple configuration and inexpensive.

As illustrated in FIG. 6B, the PMUT array 60B will be described as an example of the PMUT array 21. The PMUT array 60B includes the PMUT cells 61 and 62, the MEMS 63, through-silicon vias (TSVs) 64 and 65 and a switching circuit 66. The TSVs are electrodes that vertically penetrate through inside of a silicon semiconductor chip. The TSV 64 is electrically connected to the PMUT cell 61, penetrates through the MEMS 63 and is electrically connected to a terminal of the switching circuit 66. The TSV 65 is electrically connected to the PMUT cell 62, penetrates through the MEMS 63 and is electrically connected to the terminal of the switching circuit 66.

The switching circuit 66 is a circuit such as an LSI substrate on which connection paths of the PMUT cells 61 and 62, a switching circuit element and a pattern are formed. The switching circuit 66 controls switching of a connection of the PMUT cells 61 and 62 between a series connection and a parallel connection via the TSVs 64 and 65. In a case of the ultrasound probe 2B, the switching circuit 66 has a configuration of a CMOS circuit included in the amplifier 241 (CMOS amplifier), but this is not restrictive.

In the PMUT array 60B, a connection of the PMUT cells 61 and 62 is arbitrarily or fixedly switched between a series connection and a parallel connection by the switching circuit 66. However, the TSVs of the number corresponding to the number of the PMUT cells (the PMUT cells 61 and 62 (two)) are required. While in FIG. 6B, the TSV 64 is illustrated as one electrode, actually, one or more electrodes are required for each of the lower electrode 417 and the upper electrode 419. In a similar manner, for the TSV 65, one or more electrodes are required for each of the lower electrode 417 and the upper electrode 419.

As illustrated in FIG. 6C, the PMUT array 60C will be described as an example of the PMUT array 21. The PMUT array 60C includes the PMUT cells 61 and 62, the MEMS 63, a TSV 67 and the switching circuit 66. It is assumed that a block BL1 includes the PMUT cells 61 and 62. The PMUT array including one block BL1 having two PMUT cells will be described here to simplify description, but this is not restrictive. It is also possible to employ a configuration where the PMUT array includes a plurality of blocks or a configuration where a block includes three or more PMUT cells. In the block BL1, the PMUT cells 61 and 62 are connected in series or in parallel. In a block including three or more PMUT cells, the PMUT cells are connected in series, in parallel or in a series/parallel mixed connection.

The TSV 67 is electrically connected to the block BL1 (terminal common between the PMUT cells 61 and 62), penetrates through the MEMS 63 and is electrically connected to the terminal of the switching circuit 66. While in FIG. 6C, the TSV 67 is illustrated as one electrode, actually one or more electrodes are required for each of the lower electrode 417 and the upper electrode 419.

In the PMUT array 60C, a connection of the block BL1 is arbitrarily or fixedly switched between a series connection and a parallel connection by the switching circuit 66.

Note that it is also possible to employ a configuration where a switching circuit similar to the switching circuit 66 is provided by being connected (closely connected) in the vicinity of the PMUT cells 211 of the ultrasound probe 2A of the ultrasound diagnosis apparatus 1A. In this configuration, for example, the cable 22 includes a control line for switching circuit. Further, the controller 17A is connected to the switching circuit via a control line of the cable 22. Still further, the controller 17A controls the switching circuit via the cable 22 to switch a series/parallel mixed connection of the PMUT cells 211 (PMUT arrays 60B and 60C). Subsequently, the PMUT array 40A in the PMUT array 21 of the ultrasound probes 2A and 2B will be described with reference to FIG. 7A to FIG. 7C. An example of arrangement of the PMUT cells 211 will be described.

As illustrated in FIG. 7A, the PMUT array 40A as an example of the PMUT array 21 is a one dimensional (1D)-PMUT array of four channels. In the PMUT array 40A, a plurality of PMUT cells 50A are arranged in an orthogonal grid shape on an xy plane. A resonance frequency becomes higher as an area of the PMUT cell becomes smaller, and the resonance frequency becomes lower as the area becomes larger. Thus, a plurality of PMUT cells 50A have a single resonance frequency. Further, it is assumed that the PMUT cell 50A is a piezoelectric element that transmits/receives ultrasounds.

Further, the PMUT array 40A includes channels 401A, 402A, 403A and 404A arranged in the y axis direction (azimuth direction). In each of the channels 401A to 404A, a plurality of PMUT cells 50A are arranged. For example, scanning is performed while one clump of a plurality of channels that transmit/receive ultrasounds is sequentially shifted in a +y direction (the channel 401A to the channel 404A).

The PMUT array 40A has high sensitivity, but a frequency band is narrow. Note that in the PMUT array 40A, the PMUT cells 50A may be arranged in other forms such as a triangle grid shape.

As illustrated in FIG. 7B, the PMUT array 40B that is an example of the PMUT array 21 is a 1D-PMUT array of four channels. In the PMUT array 40B, a plurality of the PMUT cells 50B are arranged so that the number of the PMUT cells 50B becomes the same in the y axis direction on the xy plane. However, it is assumed that an area of each of the plurality of PMUT cells 50B becomes smaller toward the center side from an end portion side in the x axis direction (elevation direction). Further, it is assumed that the PMUT cell 50B is a piezoelectric element that transmits/receives ultrasounds.

Still further, the PMUT array 40B includes channels 401B, 402B, 403B and 404B arranged in the y axis direction. In each of the channels 401B to 404B, the plurality of PMUT cells 50B are arranged in the same array shape.

In the PMUT array 40B, areas of the plurality of PMUT cells 50B are different in each channel, and thus, a wide band can be achieved. To form an acoustic field, while it is preferable to arrange PMUT cells 50B having higher frequencies (smaller diameters) on an inner side (center side) in the x axis direction as in the PMUT array 40B, the arrangement is not limited to this.

As illustrated in FIG. 7C, the PMUT array 40C that is an example of the PMUT array 21 is a 1D-PMUT array of four channels. The PMUT array 40C includes a plurality of PMUT cells 50C1 as elements dedicated for transmission of ultrasounds, and a plurality of PMUT cells 50C2 as elements dedicated for reception of ultrasounds. The PMUT cells 50C1 and 50C2 are alternately arranged in an orthogonal grid shape.

Further, the PMUT array 40C includes channels 401C, 402C, 403C and 404C arranged in the y axis direction. In each of the channels 401C to 404C, the plurality of PMUT cells 50C1 and 50C2 are arranged.

In the PMUT array 40C, the number of PMUT cells 50C1 and the number of PMUT cells 50C2 correspond on a one-to-one basis within each channel, but this is not restrictive.

Subsequently, a control example of the PMUT array 70 in which connections of the PMUT cells can be switched will be described as an example of the PMUT array 60B in FIG. 6B with reference to FIG. 8A to FIG. 9B. As illustrated in FIG. 8A, the PMUT array 70 includes capacitors 71, 72, 73 and 74, switches SW1, SW2, SW3, SW4, SW5 and SW6, a power-supply terminal T1 and a grounding terminal T2.

The capacitors 71 to 74 are respectively the lower electrodes 417, the piezoelectric thin films 418 and the upper electrodes 419 of four PMUT cells represented as capacitors having electrostatic capacitance. The capacitors 71 to 74 correspond to the PMUT cells 61 and 62 (here, four) of the PMUT array 60B. The electrostatic capacitance of the capacitors 71 to 74 is respectively set as C1, C2, C3 and C4.

The switches SW1 to SW6 correspond to switches of the switching circuit 66 of the PMUT array 60B. The power-supply terminal T1 is a terminal that feeds a voltage V [V]. The grounding terminal T2 is a terminal at a ground potential 0 [V].

Here, a control example of the electrostatic capacitance of the PMUT array 70 will be described. The PMUT array 70 in FIG. 8A is in a state where all the capacitors 71 to 74 are connected in parallel (all parallel connection) by switching of the switches SW1 to SW6. Electrostatic capacitance C of the PMUT array 70 in an all parallel connection can be calculated using the following expression (1).

$$C = C 1 + C 2 + C 3 + C 4 \qquad ( 1 )$$

In particular, in a case where C1=C2=C3=C4=C0 (C0: predetermined electrostatic capacitance) in expression (1), C=4C0.

The PMUT array 70 in FIG. 8B is in a state where all the capacitors 71 to 74 are connected in series (all series connection) by switching of the switches SW1 to SW6. The electrostatic capacitance C of the PMUT array 70 in an all series connection can be calculated using the following expression (2).

$$\begin{matrix}[ Expression 1 ] & \\ C = {( \frac{1}{C 1} + \frac{1}{C 2} + \frac{1}{C 3} + \frac{1}{C 4} )}^{- 1} & ( 2 )\end{matrix}$$

In particular, in a case where C1=C2=C3=C4=C0 in expression (2), C=C0/4.

The PMUT array 70 in FIG. 9A is in a state where capacitors 71 to 74 are connected in a connection in which series connections and parallel connections are mixed (first series/parallel mixed connection) by switching of the switches SW1 to SW6. The electrostatic capacitance C of the PMUT array 70 in the first series/parallel mixed connection can be calculated using the following expression (3).

$$\begin{matrix}[ Expression 2 ] & \\ C = {( \frac{1}{C 1} + \frac{1}{C 2} + \frac{1}{C 3} + \frac{1}{C 4} )}^{- 1} & ( 3 )\end{matrix}$$

In particular, in a case where C1=C2=C3=C4=C0 in expression (3), C=(⅖)C0.

The PMUT array 70 in FIG. 9B is in a state where capacitors 71 to 74 are connected in a connection in which series connections and parallel connections are mixed (second series/parallel mixed connection) by switching of the switches SW1 to SW6. The electrostatic capacitance C of the PMUT array 70 in the second series/parallel mixed connection can be calculated using the following expression (4).

$$\begin{matrix}[ Expression 3 ] & \\ C = {( \frac{1}{C 1} + \frac{1}{C 2} + \frac{1}{C 3} + \frac{1}{C 4} )}^{- 1} & ( 4 )\end{matrix}$$

In particular, in a case where C1=C2=C3=C4=C0 in expression (4), C=C0.

In this manner, the electrostatic capacitance C of the PMUT array 70 in a series/parallel mixed connection can be adjusted by switching the switches SW1 to SW6.

Subsequently, control of weighting of transmission of ultrasounds by the PMUT array 70 will be described. Strength of transmission of ultrasounds (transmission sound pressure) corresponds to a drive voltage to be applied to the piezoelectric element, and thus can be controlled by the drive voltage. It is assumed here that a voltage (=V [V]) of the power-supply terminal T1 of the PMUT array 70 and a voltage (=0 [V]) of the grounding terminal T2 are fixed.

In the PMUT array 70 in an all parallel connection in FIG. 8A, a voltage V is applied to each of the capacitors 71 to 74.

In the PMUT array 70 in an all series connection in FIG. 8B, a voltage V/4 is applied to each of the capacitors 71 to 74.

In the PMUT array 70 in a series/parallel mixed connection in FIG. 9A, a voltage (⅖) V is applied to each of the capacitors 71 and 74. In a similar manner, a voltage V/5 is applied to each of the capacitors 72 and 73. In this manner, drive voltages can be made different for each piezoelectric element (PMUT cell). If the drive voltages are different, transmission sound pressures also become different. It is possible to control weighting of transmission of ultrasounds by utilizing this.

In the PMUT array 70 in the second series/parallel mixed connection in FIG. 9B, a voltage V/2 is applied to each of the capacitors 71 to 74.

In this manner, transmission sound pressures of ultrasounds can be adjusted by adjusting drive voltages of the PMUT array 70 in a series/parallel mixed connection by switching of the switches SW1 to SW6.

As weighting of transmission of ultrasounds, there is apodization. In the apodization, control is performed so that a transmission sound pressure of a piezoelectric element near the center>a transmission sound pressure of a piezoelectric element outside the piezoelectric element, and a side lobe is reduced. Here, the PMUT array 70A illustrated in FIG. 10 will be described. The PMUT array 70A includes the capacitors 71, 72, 73, 74 and 75, the switches SW1, SW2, SW3, SW4, SW5, SW6, SW7 and SW8, the power-supply terminal T1 and the grounding terminal T2.

In a similar manner to the capacitors 71 to 74, the capacitor 75 is the lower electrode 417, the piezoelectric thin film 418 and the upper electrode 419 of one PMUT cell represented as a capacitor having electrostatic capacitance. The capacitor 75 corresponds to the PMUT cells 61 and 62 of the PMUT array 60B. The electrostatic capacitance of the capacitor 75 is set as C5. In a similar manner to the switches SW1 to SW6, the switches SW7 and SW8 correspond to a switch of the switching circuit 66 of the PMUT array 60B.

A case will be considered where the capacitors 71 to 75 are sequentially arranged in the azimuth direction or in the elevation direction in the PMUT array 70A. The PMUT array 70A is in a state where the capacitors 71 to 75 are connected in a connection state in which series connections and parallel connections are mixed (third series/parallel mixed connection) by switching of the switches SW1 to SW8. In the PMUT array 70A, a voltage V/4 is applied to each of the capacitors 71, 72, 74 and 75. In a similar manner, a voltage V/2 is applied to the capacitor 73.

In the PMUT array 70A, a drive voltage of the capacitor 73 near the center in the arrangement direction of the capacitors 71 to 75>drive voltages of the capacitors 71, 72, 74 and 75 outside in the arrangement direction of the capacitors 71 to 75. By setting the arrangement direction of the capacitors 71 to 75 as a direction of apodization, the PMUT array 70A can be applied to the apodization.

Subsequently, a favorable ratio of series connections and parallel connections of the PMUT cells of the PMUT array 80 within one channel in the PMUT array having a plurality of channels that is an example of the PMUT array 21 of the ultrasound probes 2A and 2B will be described with reference to FIG. 11.

The PMUT array 80 includes a plurality of capacitors 81 of the PMUT array. The capacitors 81 are respectively the lower electrodes 417, the piezoelectric thin films 418 and the upper electrodes 419 of the PMUT cells represented as capacitors having electrostatic capacitance.

In the PMUT array 80, a plurality of capacitors 81 connected in series are connected in parallel. In the PMUT array 80 of one channel, the number of the capacitors 81 connected in series (the number of series connections/channel) is set as the number of series connections Ns. The number of the capacitors 81 connected in parallel in the PMUT array 80 of one channel (the number of parallel connections/channel) is set as the number of parallel connections Np. Further, the electrostatic capacitance of each capacitor 81 is set as electrostatic capacitance C0. Still further, a drive voltage to be applied to the whole PMUT array 80 is set as a voltage Vin, and a drive voltage to be applied to each capacitor 81 is set as a voltage V0.

Next, as indicated in Table 1, Comparative Examples 1 and 2 and Examples 1, 2 and 3 in a case where the number of the capacitors 81 for each channel (the number of cells/channel) is set at 200 in the PMUT array 80, will be described.

TABLE 1

| | Comparative | | | | Comparative |
|---|---|---|---|---|---|
| | Example 1 | Example 1 | Example 2 | Example 3 | Example 2 |
| The number of | 200 | 200 | 200 | 200 | 200 |
| cells/channel | | | | | |
| The number of parallel | 200 | 100 | 50 | 25 | 1 |
| connections/channel (=Np) | | | | | |
| The number of series | 1 | 2 | 4 | 8 | 200 |
| connections/channel (=Ns) | | | | | |
| Cch[pf] | 740 | 185 | 46 | 11 | 0.02 |
| Z[Ω](3 MHz) | 72 | 287 | 1147 | 4587 | 2866767 |
| Z[Ω](5 MHz) | 43 | 172 | 688 | 2752 | 1720060 |
| Z[Ω](10 MHz) | 22 | 86 | 344 | 1376 | 860030 |
| Z[Ω](15 MHz) | 14 | 57 | 229 | 917 | 573353 |
| Vo/Vin | 1.0 | 0.500 | 0.250 | 0.125 | 0.005 |

Comparative Example 1 employs a configuration where all the capacitors 81 of the PMUT array 80 are connected in parallel. Comparative Example 2 employs a configuration where all the capacitors 81 of the PMUT array 80 are connected in series. Examples 1 to 3 employ a configuration where the capacitors 81 of the PMUT array 80 are connected in a connection in which series connections and parallel connections are mixed.

In the PMUT array 80 of one channel, the whole electrostatic capacitance is set as electrostatic capacitance Cch. The electrostatic capacitance Cch can be calculated using the following expression. Cch=C0×Np/Ns

Further, impedance Z of the PMUT array 80 in transmission of ultrasounds of 3 MHz is set as Z [Ω] (3 MHZ). The impedance Z of the PMUT array 80 in transmission of ultrasounds of 5 MHz is set as Z [Ω] (5 MHz). The impedance Z of the PMUT array 80 in transmission of ultrasounds of 10 MHz is set as Z [Ω] (10 MHz). The impedance Z of the PMUT array 80 in transmission/reception of ultrasounds of 15 MHz is set as Z [Ω] (15 MHz). Voltage efficiency of the PMUT array 80 upon transmission of ultrasounds is set as V0/Vin.

However, the impedance Z is set at the impedance Z=1 (ωC), and a case where C is dominant is assumed. In other words, the impedance Z can be adjusted by adjusting the electrostatic capacitance Cch.

From respective values in Table 1, image quality, cost, transmission sensitivity, heat generation, and the like, of the ultrasound diagnosis apparatus, Comparative Example 1 and Example 1 including the cable 22 are suitable for the ultrasound diagnosis apparatus 1A including the transmitter/receiver 12. Further, Example 1 in which the impedance Z is greater is more preferable than Comparative Example 1 in which the impedance Z is smaller. In a similar manner, Examples 2 and 3 in which the impedance Z can be made smaller than Comparative Example 2 in which the impedance Z is too great, are suitable for the ultrasound diagnosis apparatus 1B including the transmitter/receiver 24 within the ultrasound probe 2B.

Further, while in Examples 1 to 3 in a series/parallel mixed connection, transmission efficiency based on voltage efficiency V0/Vin upon transmission of ultrasounds decreases compared to Comparative Example 1, it is only necessary to perform correction driving using a transmission power supply.

Further, the configuration of the PMUT array 40C in FIG. 7C can be applied to the configuration of the PMUT array 60A in FIG. 6A (configuration where a connection cannot be switched between a series connection and a parallel connection). In this case, the number of parallel connections Np is preferably different from the number of series connections Ns in a series/parallel mixed connection of the PMUT cells 50C1 dedicated for transmission and the PMUT cells 50C2 dedicated for reception. The impedance Z of the PMUT cells 50C1 for reception is adjusted to be high (the CMOS amplifier is assumed), and the impedance Z of the PMUT cells 50C2 for transmission is adjusted to be low (emphasis is placed on transmission efficiency).

Further, the configuration of the PMUT array 40C can be applied to the configuration of the PMUT arrays 60B and 60C in FIG. 6B and FIG. 6C (configuration where a connection can be switched between a series connection and a parallel connection). In this configuration, the impedance Z of the PMUT cells is adjusted to be high (the CMOS amplifier is assumed) upon reception, and the impedance Z of the PMUT cells is adjusted to be low (emphasis is placed on transmission efficiency) upon transmission.

As described above, according to the present embodiment, the ultrasound probes 2A and 2B include the PMUT array 21 including a plurality of channels and including a plurality of three or more PMUT cells 211 within each channel. The piezoelectric thin films 418 of the plurality of PMUT cells 211 are electrically connected to each other in a series/parallel mixed connection in which parallel connections and series connections are mixed.

The ultrasound diagnosis apparatus 1A includes the ultrasound probe 2A, the transmitter/receiver 12 that generates a drive signal for transmission of ultrasounds, outputs the drive signal to the PMUT cells 211 of the PMUT array 21 and receives a reception signal of the ultrasounds from the PMUT cells 211 of the PMUT array 21, and an image generator 13 that generates ultrasonic image data on the basis of the reception signal input from the ultrasound probe 2A.

The ultrasound probe 2A includes the transmitter/receiver 24 that generates a drive signal for transmission of ultrasounds, outputs the drive signal to the PMUT cells 211 of the PMUT array 21 and receives a reception signal of ultrasounds from the PMUT cells 211 of the PMUT array 21. The ultrasound diagnosis apparatus 1B includes the ultrasound probe 2B and the image generator 13 that generates ultrasonic image data on the basis of the reception signal input from the transmitter/receiver 24.

It is therefore possible to appropriately control electrostatic capacitance of the piezoelectric thin films 418 of the PMUT cells 211 in accordance with a connection form between the ultrasound probes 2A and 2B and parts in subsequent stages (the ultrasound diagnosis apparatus bodies 10A and 10B). The connection form is whether or not the cable 22 exists, a position of the transmitter/receiver (amplifiers 121 and 221), and the like. Impedance matching can be achieved by this control of the electrostatic capacitance. In particular, electrical matching between the piezoelectric elements (piezoelectric thin films 418) of the PMUT cells and the transmitter/receivers 12 and 22 upon reception of ultrasounds is improved, so that reception sensitivity and a signal to noise ratio (SNR) can be improved. This results in improving image quality of ultrasonic image data, which can improve diagnosis easiness.

Further, the plurality of PMUT cells 50B of the PMUT array 40B in FIG. 7B include PMUT cells having resonance frequencies different from each other. It is therefore possible to achieve a wider band of the ultrasound probes 2A and 2B.

Further, the PMUT arrays 60A to 60C in FIG. 6A to FIG. 6C and the PMUT array 40C in FIG. 7C are designed or switching is controlled so that the number of series connections and the number of parallel connections in a series/parallel mixed connection become different between upon transmission and upon reception of ultrasounds. It is therefore possible to optimize impedance matching between upon transmission and upon reception of ultrasounds.

Further, in the PMUT array 40C in FIGS. 7A-FIG. 7C, the plurality of PMUT cells 211 include the PMUT cells 50C1 dedicated for transmission of ultrasounds and the PMUT cells 50C2 dedicated for reception of ultrasounds. This makes it possible to constitute transmission/reception separated type PMUT cells 211, so that it is possible to easily perform control for optimizing impedance matching between upon transmission and upon reception of ultrasounds.

Further, the ultrasound probes 2A and 2B include the switching circuit 66 connected to the PMUT cells 61 and 62 of the PMUT array 60B in FIG. 6B via the TSVs 64 and 65. The switching circuit 66 switches a connection of at least one of a series connection or a parallel connection of the PMUT cells 61 and 62. To implement this, impedance is adjusted to some extent within the MEMS substrate, and switching of connection is made variable at the switching circuit 66. This makes it possible to appropriately adjust impedance for each transmission/reception, for each scanning mode and for each utilization scene. This scanning mode is a scanning mode such as a B mode, a color Doppler mode, a PWD and a harmonic. The utilization scene is a scene of scanning in which at least one of impedance or a transmission sound pressure is different in accordance with a predetermined parameter. An example of the utilization scene will be described later as a modified example.

Further, the switching circuit 66 switches connections of the PMUT cells of the PMUT array for each block including a plurality of PMUT cells. It is therefore possible to reduce the number of TSVs by using a block of the PMUT cells as a control unit.

### Application Example

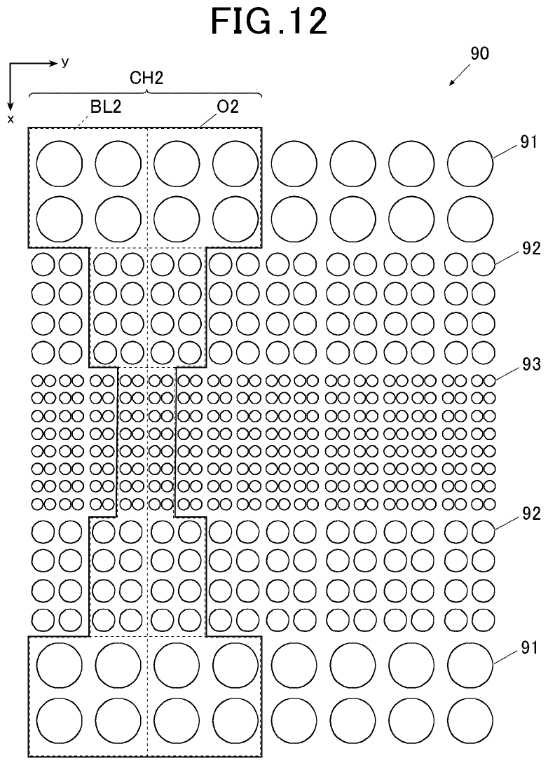

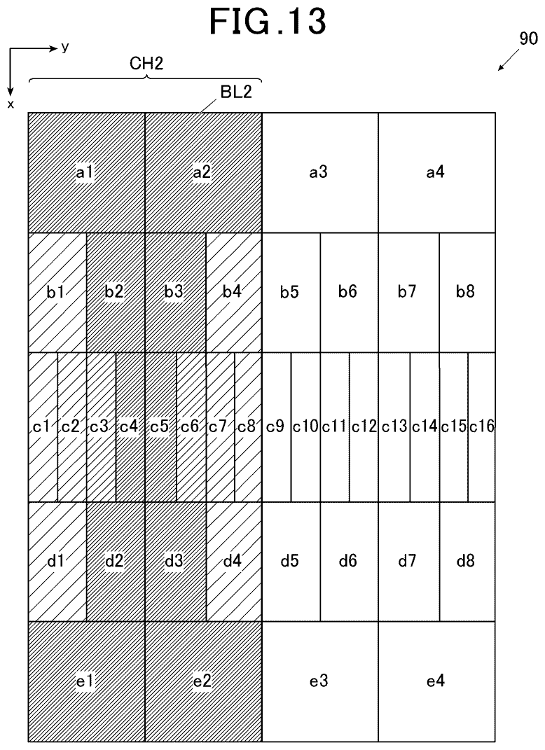

An application example of the ultrasound diagnosis apparatuses 1A and 1B of the above-described embodiment will be described with reference to FIG. 12 and FIG. 13. FIG. 12 is a schematic plan view illustrating PMUT cells 91, 92 and 93 of a PMUT array 90. FIG. 13 is a schematic plan view illustrating a block BL2 of the PMUT array 90.

The PMUT array 90 that is an example of the PMUT array 21 of the ultrasound probes 2A and 2B of the ultrasound diagnosis apparatuses 1A and 1B of the above-described embodiment will be described. The PMUT array 90 has a configuration in which application is further added to the PMUT array 40B in FIG. 7B. Note that it is assumed that the impedance Z is appropriately adjusted through adjustment of a series/parallel mixed connection in the PMUT array 90, but this is not restrictive.

The PMUT array 90 is a 1D-PMUT array and includes a plurality of PMUT cells 91, a plurality of PMUT cells 92 and a plurality of PMUT cells 93. The PMUT cells 93 with a relatively small diameter are arranged in a center region in the x axis direction (elevation direction) of the PMUT array 90. The PMUT cells 92 with a larger diameter than that of the PMUT cells 93 are arranged in a region outside in the x axis direction of the PMUT cells 93 of the PMUT array 90. The PMUT cells 91 with a larger diameter than that of the PMUT cells 92 are arranged in a region outside in the x axis direction of the PMUT cells 92 of the PMUT array 90. In the PMUT array 90, there is a relationship of a resonance frequency of the PMUT cells 93>a resonance frequency of the PMUT cells 92>a resonance frequency of the PMUT cells 91.

A channel of the PMUT array 90 is set as a channel CH2, a transmission opening within the channel CH2 is set as an opening O2, and the opening O2 is indicated with a solid line in FIG. 12. The opening O2 includes a plurality of blocks BL2, and the blocks BL2 are indicated with a dotted line in FIG. 12. Here, as illustrated in FIG. 13, the plurality of PMUT cells 91, 92 and 93 of the PMUT array 90 are divided into a plurality of blocks BL2. The blocks BL2 include blocks a1 to a4, blocks b1 to b8, blocks c1 to c16, blocks d1 to d8 and blocks e1 to e4.

Each of the blocks a1 to a4 and e1 to e4 includes four PMUT cells 91. Each of the blocks b1 to b8 and d1 to d8 includes eight PMUT cells 92. Each of the blocks c1 to c16 includes 16 PMUT cells 93. The PMUT array 60C in FIG. 6C is applied to control of drive voltages of all the PMUT cells within each block BL2. The same common drive voltage is applied to the PMUT cells by one TSV 67 within each block BL2 by the switching circuit 66.

In the PMUT array 90 of the ultrasound probes 2A and 2B, a series of a plurality of channels CH2 that transmit/receive ultrasounds are shifted in block units in the +y direction (azimuth direction). By this means, two-dimensional ultrasonic image data is scanned. Within one channel CH2, resonance frequencies of the PMUT cells 91, 92 and 93 are distributed in the x axis direction to achieve a wider band. Further, in the PMUT array 90, the PMUT cells 91 of a low frequency are disposed at an end portion in the x axis direction, and the PMUT cells 93 of a high frequency are disposed at a center portion in the x axis direction. An element opening width (azimuth pitch) of one channel is different (roughly equal to or less than one wavelength) in accordance with a frequency, and thus, a pitch of high frequency elements is widened at a simple rectangular opening, and a side lobe occurs.

Thus, in the channel CH2, a drive voltage of the opening O2 is controlled to be high by the switching circuit 66. More specifically, concerning the drive voltages of the blocks c1 to c8, the drive voltages are weighted so that the drive voltage of the block c1<the drive voltage of the block c2<the drive voltage of the block c3<the drive voltage of the block c4=the drive voltage of the block c5>the drive voltage of the block c6>the drive voltage of the block c7>a drive voltage of the block c8. In a similar manner, concerning the drive voltages of the blocks b1 to b4, the drive voltages are weighted so that the drive voltage of the block b1<the drive voltage of the block b2=the drive voltage of the block b3>the drive voltage of the block b4. In a similar manner, concerning the drive voltages of the blocks d1 to d4, the drive voltages are weighted so that the drive voltage of the block d1<the drive voltage of the block d2=the drive voltage of the block d3>the drive voltage of the block d4.

In a similar manner, concerning the drive voltages of the blocks a1 and a2, the drive voltages are weighted so that the drive voltage of the block a1=the drive voltage of the block a2. In a similar manner, concerning the drive voltages of the blocks e1 and e2, the drive voltages are weighted so that the drive voltage of the block e1=the drive voltage of the block e2. By weighting of these drive voltage, it is possible to weight (apodize) sound pressure distributions upon transmission of ultrasounds in the y axis direction and prevent a side lobe in the y axis direction.

Further, concerning the drive voltages of the blocks a1, b2, c4, d2 and e1, the drive voltages are weighted so that the drive voltage of the block c4>the drive voltage of the block b2=the drive voltage of the block d2>the drive voltage of the block a1=the drive voltage of the block e1. By weighting of these drive voltages, it is possible to weight (apodize) sound pressure distributions upon transmission of ultrasounds in the x axis direction and prevent a side lobe in the x axis direction.

As described above, according to the present application example, the series/parallel mixed connection of the PMUT array 90 is a connection in which the drive voltages of the PMUT cells 91 to 93 are different between on the end portion side and on the center side in the x axis direction (elevation direction) of the opening O2 of the PMUT array 90. Specifically, the drive voltage of the PMUT cells 93 is made greater, and the drive voltage of the PMUT cells 91 is made smaller. This can weight transmission sound pressures of ultrasounds in the elevation direction, so that it is possible to prevent a side lobe in the elevation direction by an apodization effect.

Further, the series/parallel mixed connection of the PMUT array 90 is a connection in which the drive voltages of the PMUT cells 91 to 93 are different between on the end portion side and on the center side in the y axis direction (azimuth direction) of the opening O2 of the PMUT array 90. Specifically, the drive voltage of the PMUT cells 91 to 93 on the center side in the azimuth direction is made greater, and the drive voltage of the PMUT cells 91 to 93 on the end portion side is made smaller. This can weight transmission sound pressures of ultrasounds in the azimuth direction, so that it is possible to prevent a side lobe in the azimuth direction by an apodization effect.

### Modified Example

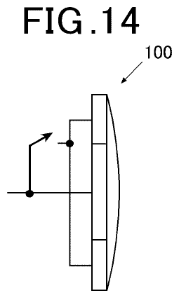

A modified example of the above-described embodiment will be described with reference to FIG. 14. FIG. 14 is a schematic view illustrating the PMUT array 100.

In the above-described embodiment, a configuration has been described where the 1D-PMUT array is applied to the PMUT array 21 of the ultrasound probes 2A and 2B of the ultrasound diagnosis apparatuses 1A and 1B. In the present modified example, a configuration will be described where a PMUT array 100 of a 1.25D-PMUT array illustrated in FIG. 14 is applied to the PMUT array 21. In this configuration, a scene in which the impedance Z of the PMUT array 100 is adjusted in accordance with a drawing depth that is a predetermined parameter is employed as a utilization scene.

The 1.25D-PMUT array is a PMUT array that variably adjusts a length of the opening in the elevation direction (elevation opening) in accordance with the drawing depth. Specifically, the elevation opening is made greater at a portion where the drawing depth is deep and made smaller at a portion where the drawing depth is shallow.

If the elevation opening changes in the 1.25D-PMUT array in an all parallel connection as in the related art, impedance of the PMUT cells changes, an electrical matching condition changes, and sensitivity changes. This results in a problem that sensitivity unevenness occurs in an ultrasonic image.

In the present modified example, the PMUT array 100 of the 1.25D-PMUT array in which a connection of the PMUT cells in a series/parallel mixed connection can be switched is applied to the PMUT array 21. It is assumed that as a configuration for switching a connection of the PMUT cells, a switching configuration similar to that of the PMUT array 60B in FIG. 6B or that of the PMUT array 60C in FIG. 6C is applied to the PMUT array 100.

Then, a series/parallel mixed connection state of the PMUT cells is switched and controlled by the switching circuit in accordance with the elevation opening, so that a width of the impedance is made as small as possible. More specifically, for example, a 1.25D-PMUT array for 10 MHz is applied to the PMUT array 100. The drawing depth is switched in three stages for a deep portion (opening of 6 mm), for a mid-depth portion (opening of 3 mm) and for a shallow portion (opening of 1 mm). As indicated in Table 2 next, configuration examples of the elevation openings in three stages applying the 1.25D-PMUT array for 10 MHz in an all parallel connection are set as Comparative Examples 3, 4 and 5. Configurations of the elevation openings in three stages applying the PMUT array 100 of the 1.25D-PMUT array for 10 MHz in a series/parallel mixed connection are set as Examples 4, 5 and 6.

TABLE 2

| | | | | Series/parallel mixed | | |
|---|---|---|---|---|---|---|
| | All parallel connections | | | connection | | |
| | Comparative | Comparative | Comparative | Example | Example | Example |
| | Example 3 | Example 4 | Example 5 | 4 | 5 | 6 |
| Elevation opening | 6 (for | 3 (for | 1 (for | 6 (for | 3 (for | 1 (for |
| [mm] | deep | mid-depth | shallow | deep | mid-depth | shallow |
| | portion) | portion) | portion) | portion) | portion) | portion) |
| The number of | 420 | 210 | 70 | 420 | 210 | 70 |
| cells/channel | | | | | | |
| The number of | 420 | 210 | 70 | 140 | 105 | 70 |
| parallel | | | | | | |
| connections/channel | | | | | | |
| (=Np) | | | | | | |
| The number of | 1 | 1 | 1 | 3 | 2 | 1 |
| series | | | | | | |
| connections/channel | | | | | | |
| (=Ns) | | | | | | |
| Cch[pf] | 1554 | 777 | 259 | 172 | 194 | 259 |
| Z[Ω](10 MHz) | 10 | 20 | 61 | 92 | 82 | 61 |

It is assumed that the number of capacitors (the number of cells) for each channel of a plurality of PMUT cells of the 1.25D-PMUT array, the number of parallel connections Np, the number of series connections Ns, the electrostatic capacitance Cch [pf] and the impedance Z (10 MHz) are similar to those of the PMUT array 80 in FIG. 11. In Comparative Examples 3 to 5, the impedance Z (10 MHz)=10 to 61[Ω], and an impedance difference (sensitivity difference) is large which is up to approximately six times.

In contrast, in Examples 4 to 6, the impedance Z (10 MHz)=61 to 92[Ω], and an impedance difference (sensitivity difference) is small which is up to approximately 1.5 times.

As described above, according to the present modified example, in the PMUT array 100, the switching circuit switches a connection of the PMUT cells in a series/parallel mixed connection in accordance with the drawing depth (elevation opening). The impedance is adjusted by switching a connection of the PMUT cells. It is therefore possible to reduce an impedance difference (sensitivity difference) by change of the elevation opening, so that it is possible to reduce sensitivity unevenness of ultrasonic image data.

Note that description in the above-described embodiment, application example and modified example merely discloses one preferred example of the ultrasound probe and the ultrasound diagnosis apparatus according to the present invention, so that the description is not restrictive. For example, it is also possible to employ a configuration in which at least two of the above-described embodiment, application example or modified example are combined as appropriate.

Further, in the above-described modified example, a case has been described where the utilization scene is a scene in which the impedance of the PMUT array is different in accordance with the elevation opening that is a predetermined parameter. Further a configuration is employed where in this utilization scene, the switching circuit switches a connection of the PMUT cells in a series/parallel mixed connection in accordance with the elevation opening (drawing depth) to appropriately adjust the impedance. However, the present invention is not limited to this configuration. It is also possible to employ a configuration where the switching circuit switches a connection of the PMUT cells in a series/parallel mixed connection in accordance with a scanning mode or a predetermined parameter. At least one of the impedance or the transmission sound pressures (drive voltages) of the PMUT array is appropriately adjusted by switching of the connection of the PMUT cells in the series/parallel mixed connection. As the predetermined parameter, other parameters such as a frequency band of ultrasounds may be used. The scanning mode is an arbitrary scanning mode described in the above-described embodiment. For example, in a case where B mode image data and color Doppler image data are displayed in a superimposed manner, the switching circuit appropriately switches the connection of the PMUT cells in the series/parallel mixed connection upon transmission/reception of ultrasounds in each mode.

Further, detailed configurations and detailed operation of respective components constituting the ultrasound diagnosis apparatuses in the above-described embodiment, application example and modified example can be changed as appropriate within a range not deviating from the gist of the present invention.

Although embodiments of the present invention have been described and illustrated in detail, the disclosed embodiments are made for purposes of illustration and example only and not limitation. The scope of the present invention should be interpreted by terms of the appended claims.

What is claimed is:

1. An ultrasound probe comprising:
a piezoelectric micromachined ultrasonic transducer (PMUT) array including a plurality of channels and including a plurality of three or more PMUT cells within each of the channels,
wherein piezoelectric elements of the plurality of PMUT cells are electrically connected to each other in a series/parallel mixed connection in which parallel connections and series connections are mixed.

2. The ultrasound probe according to claim 1, wherein the plurality of PMUT cells include PMUT cells having resonance frequencies different from each other.

3. The ultrasound probe according to claim 1, wherein the series/parallel mixed connection is a connection in which drive voltages of the PMUT cells are different between on an end portion side and on a center side in an elevation direction of a transmission opening of the PMUT array.

4. The ultrasound probe according to claim 1, wherein the series/parallel mixed connection is a connection in which drive voltages of the PMUT cells are different between on an end portion side and on a center side in an azimuth direction of a transmission opening of the PMUT array.

5. The ultrasound probe according to claim 1, wherein a number of series connections and a number of parallel connections of the series/parallel mixed connection are different between upon transmission and upon reception of ultrasounds.

6. The ultrasound probe according to claim 1, wherein the plurality of PMUT cells include PMUT cells dedicated for transmission of ultrasounds and PMUT cells dedicated for reception of ultrasounds.

7. The ultrasound probe according to claim 1, further comprising:
a switching circuit that is connected to the PMUT cells of the PMUT array via electrodes and switches at least one connection among series connections and parallel connections of the PMUT cells.

8. The ultrasound probe according to claim 7, wherein the switching circuit switches a connection of the PMUT cells for each block including a plurality of PMUT cells.

9. The ultrasound probe according to claim 7, wherein the switching circuit switches a connection of the PMUT array in accordance with a scanning mode or a predetermined parameter.

10. The ultrasound probe according to claim 1, further comprising:
a transmitter/receiver that generates a drive signal for transmission of ultrasounds and outputs the drive signal to the PMUT cells of the PMUT array and receives a reception signal of ultrasounds from the PMUT cells of the PMUT array.

11. An ultrasound diagnosis apparatus comprising:
the ultrasound probe according to claim 1; and
an image generator that generates ultrasonic image data on a basis of a reception signal input from the ultrasound probe.

12. An ultrasound diagnosis apparatus comprising:
the ultrasound probe according to claim 1;
a transmitter/receiver that generates a drive signal for transmission of ultrasounds and outputs the drive signal to the PMUT cells of the PMUT array, and receives a reception signal of ultrasounds from the PMUT cells of the PMUT array; and
an image generator that generates ultrasonic image data on a basis of a reception signal input from the transmitter/receiver.
